(19) United States
(12) Patent Application Publication
(10) Pub. No.: US 2022/0031255 A1
(43) Pub. Date: Feb. 3, 2022

(54) METHOD AND SYSTEM FOR HEALTH IMPROVEMENT USING TOILET SEAT SENSORS

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/390,826

(22) Filed: Jul. 30, 2021

Related U.S. Application Data
(60) Provisional application No. 63/103,412, filed on Aug. 3, 2020.

(51) Int. Cl. A61B 5/00; A47K 13/24; A61B 5/024; A61B 5/11; A61B 5/1455; A61B 5/318; A61B 5/117; A61B 5/0205; A61B 5/021; A61M 37/00; G16B 30/00; G16B 20/20

(52) U.S. Cl. CPC A61B 5/7282; A61M 2037/0023; A61B 5/6887; A61B 5/746; A61B 5/7221; A61B 5/02416; A61B 5/1102; A61B 5/14551; A61B 5/318; A61B 5/4833; A61B 5/117; A61B 5/4839; A61B 5/0205; A61B 5/021; A61M 37/0015; G16B 30/00; G16B 20/20; A61B 2562/0204; A47K 13/24

(57) ABSTRACT

In a telemedicine system, physiological sensors are provided in a toilet seat for monitoring physiological parameters of the subject, corroborate sensor data against other sensor data and notify personnel in the event of a problem or Concern.

### FIELD OF THE INVENTION

The present invention relates to health monitoring, diagnosis and treatment of people. In particular it relates to the use of electronic sensors and devices for remote medicine.

### BACKGROUND OF THE INVENTION

As health costs rise, the idea of telemedicine or remote medicine is becoming increasingly attractive.

However, most telemedicine solutions offered to date are rather rudimentary, often comprising simply a video conferencing facility to permit a user to visually interact with a doctor. With such systems the physician is limited in his or her diagnosis by the symptoms that the patient can verbalize, and by the video image that the physician can see on the screen.

It would therefore be beneficial to supplement this information with data gathered by sensors.

However, attaching blood pressure cuffs or capturing blood samples are activities that not all people can readily accomplish. It would therefore be desirable to have a telemedicine system that is ambient in nature, in the sense that it requires no proactive tasks on the part of the patient or user and is not carried around with the user.

### SUMMARY OF THE INVENTION

According the invention there is provided a system for remote medicine, comprising a smart toilet seat that includes at least one toilet seat sensor; at least one non-toilet seat sensor; a processor connected to the sensors; memory enabled with machine readable code defining an algorithm for identifying a trigger event based on anomalies in the data from at least one toilet seat sensor, and defining an algorithm for corroborating the trigger event from the at least one toilet seat sensor with data from at least one other toilet seat sensor or at least one non-toilet seat sensor, to define a flagging event, and means for notifying one or more people about the flagging event and details about said event.

An anomaly may include changes in physiological parameters, and characteristics or conditions of interest.

The at least one toilet seat sensor may include one or more of, an electrocardiogram (ECG) for measuring the electrical activity of the heart, a pulse oximeter or photoplethysmogram (PPG) for measuring blood oxygenation and localized pulse timing, a ballistocardiogram (BCG) for measuring the mechanical forces associated with the cardiac cycle, and a body weight sensor.

The at least one non-toilet seat sensor may include one or more of an image capture device, a microphone, a genetic sensor, and a drug compliance sensor.

The image capture device may include one or more cameras mounted at different locations and operating at different ranges of the frequency spectrum. The at least one non-toilet seat sensor may include a camera mounted in the toilet for subject authentication.

The algorithm for corroborating a trigger event may include logic for time stamping data from the sensors, wherein the corroboration of data includes an AI system configured to identify trigger events in the data from one or more of the sensors and comparing to data from one or more of the other sensors for a corresponding time frame.

The smart toilet seat may further include at least one of, means for administering medication or drugs to the subject, and means for capturing at least one of genetic data and drug compliance data from the subject. For example, the administration of medication may comprise administration using retractable micro-needles in the toilet seat or a jelly or cream housed in a housing in the toilet seat with an active ingredient that is ejected to the surface of the seat through a micro-pore array, and is dermally absorbable.

Further, according to the invention, there is provided a method of maintaining or improving the health of a subject, comprising providing a subject's toilet with a smart toilet seat that includes one or more sensors for monitoring the physiological or psychological condition of the subject; providing the subject or their living quarters with one or more sensors for monitoring the physiological or psychological condition of the subject; detecting a trigger event in the data from at least one of the sensors based on anomalies compared to historical data from said subject or from other subjects, and comparing the trigger events to data from one or more of the other sensors to provide corroboration of a trigger event from at least one other sensor, and notifying one or more persons of a corroborated trigger event (also referred to herein as a flagging event).

Comparing the trigger event to data from at least one of the other sensors may include identifying the time frame of the trigger event and analyzing the data from at least one of the other sensors over a corresponding time frame or relevant adjacent timeframe.

For instance, the explanation for a change in heart rate, heart pressure, or blood oxygen perfusion may be provided by data showing exposure by the subject to certain foods, drugs, or physical exercise in an adjacent timeframe.

The method may further include capturing genetic data from the subject while the subject is sitting on the toilet seat, and may further comprise administering medication to the subject while the subject is sitting on the toilet seat.

The method may further include analysis of the genetic data such as genomic sequencing, or attempting to identify specific genetic markers or sets of markers indicative of genetic predispositions, reactions or epigenetic changes in response to treatment(s).

Still further, according to the invention, there is provided a method of providing telehealth to a subject, comprising detecting physiological parameters of a subject using at least two sensors operating according to different modalities; comparing the parameters of each sensor to previously captured data obtained from the subject to identify changes in the data (trigger event); determining whether the changes exceed a pre-defined threshold to define a flagging event, and comparing the trigger event identified from data from one sensor, which do not rise to the level of a flagging event, to data from at least one other sensor for a corresponding time frame; defining a flagging event based on corroboration of a trigger event, and notifying one or more of: the subject, a designated contact person, supervisory personnel, and a physician in case of a flaggable event. At least one of the sensors may be mounted on the subject's toilet seat. Other sensors may be mounted on the toilet seat, on the toilet, or in the subject's home, or may be sensors carried by the subject.

The physiological parameters may include at least one of patient weight, heart-related data, blood oxygenation data, and genetic data, wherein the heart-related data may include one or more of blood pressure, heart rate, and Electrocardiogram (ECG) data.

The heart-related data and weight may be detected while the subject is sitting on a toilet, using sensors provided on the toilet seat.

The identifying of changes and comparing of sensor data may include the use of an artificial intelligence (AI) system.

The method may further include verifying the identity of the subject.

The method may further comprise administering medication to the subject while the subject is sitting on the toilet.

### BRIEF DESCRIPTION OF THE DRAWINGS

FIG. 1 is a three-dimensional depiction of one embodiment of a system of the invention;

FIG. 2 is a flow chart defining the logic of one embodiment of an anomaly detection algorithm implemented in an AI system;

FIG. 3 is a flow chart defining the logic of one embodiment of an anomaly detection and corroboration algorithm implemented in an AI system;

FIG. 4 is one embodiment of an overview dashboard page of the invention, and

FIG. 5 is one embodiment of a subject-specific dashboard page of the invention.

### DETAILED DESCRIPTION OF THE INVENTION

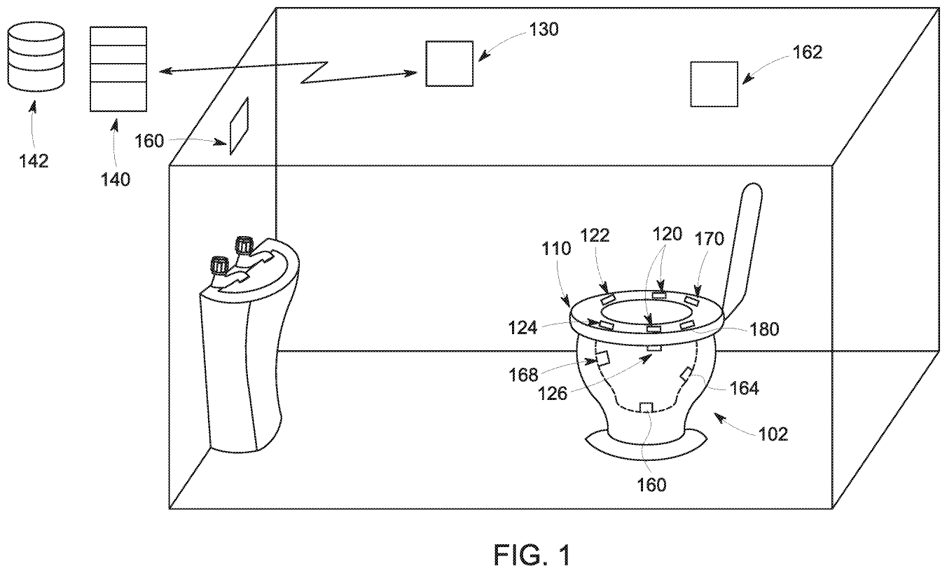

One embodiment of a system of the present invention is shown in FIG. 1, which shows a bathroom 100 with a toilet 102. The toilet includes a toilet seat 110.

The toilet seat in this embodiment includes 4 sets of sensors for measuring different physiological parameters of a subject sitting on the seat 110: an electrocardiogram (ECG) 120 for measuring the electrical activity of the heart, a photoplethysmogram (PPG) 122 for measuring blood oxygenation and localized pulse timing, a ballistocardiogram (BCG) 124 for measuring the mechanical forces associated with the cardiac cycle, and a body weight sensor 126.

The sensors 120, 122, 124, 126 are provided with short range communication means (in this case, Bluetooth) for transmitting their data to a communications hub 130, which includes cell phone and internet communications means for communicating with an external processor and memory, which in this embodiment comprises a server 140 and database 142. In addition to storing physiology data for the subject, including ECG, PPG, BCG and weight data of the subject, in order to define base values for the subject, the database 142 captures sensor data from the sensors 120, 122, 124, 126 whenever the subject sits on the toilet seat 110.

The server 140 includes memory that is configured with machine readable code that defines an algorithm for identifying a trigger event based on anomalies or changes in the data from at least one of said sensors 120, 122, 124, 126 compared to its corresponding base value.

The server 140 also includes machine readable code defining an algorithm for corroborating a flagging event from the at least one toilet seat sensor with data from at least one other toilet seat sensor or from at least one non-toilet seat sensor (which are discussed further below).

In the event of a deviation in any of the sensor data by a predefined first amount relative to the base data (also referred to herein as an anomaly or a trigger event), which is corroborated by data from at least one other sensor for the same time-frame or related time-frame, or if any sensor data deviates from its base value by a second predefined amount that is greater than the first predefined amount, the anomaly is defined as a flaggable event, and the server 140 notifies one or more persons associated with emergency numbers or other contact method and information stored in the database 142.

In the present embodiment, the system is implemented as part of the bathroom of a resident of a continuous care retirement community (CCRC). The system includes a web site stored on a server, e.g., the server 140. The server 140 also defines a dashboard accessible by support personnel at the CCRC and by an emergency response team that can notify first responders or relatives of the subject, or can provide health information directly to the subject. It will be appreciated that the access capabilities and data available to support personnel will be different to that provided to a subject. Support personnel may be able to access data for all persons under their supervision, while a subject may only be able to access his or her own data.

As indicated above, the present invention involves identification and analysis of anomalies. In one embodiment, the anomaly analysis is implemented in software and involves logic in the form of machine readable code defining an algorithm or implemented in an artificial intelligence (AI) system, which is stored on a local or remote memory (as discussed above), and which defines the logic used by a processor to perform the analysis and make assessments.

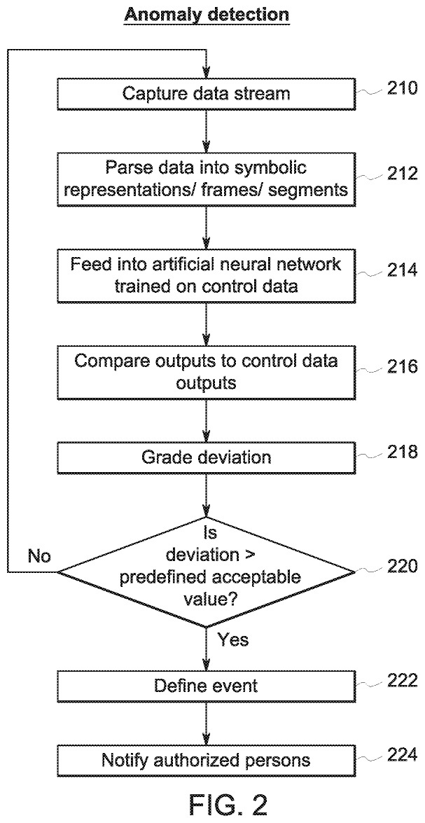

One such embodiment of the logic based on grading the level of the anomaly to determine if it surpasses a predefined threshold, is shown in FIG. 2, which defines the analysis based on sensor data that is evaluated by an Artificial Intelligence (AI) system, in this case an artificial neural network. Data from a sensor is captured (step 210) and is parsed into segments (also referred to as symbolic representations or frames) (step 212). The symbolic representations are fed into an artificial neural network (step 214), which has been trained based on control data (e.g. similar previous events involving the same party or parties or similar third-party events). The outputs from the AI are compared to outputs from the control data (step 216) and the degree of deviation is graded in step 218 by assigning a grading number to the degree of deviation. In step 220 a determination is made whether the deviation exceeds a predefined threshold, in which case the anomaly is registered as an event (step 222) and one or more authorized persons is notified (step 224) if the event qualifies as an emergency event based on the grading number.

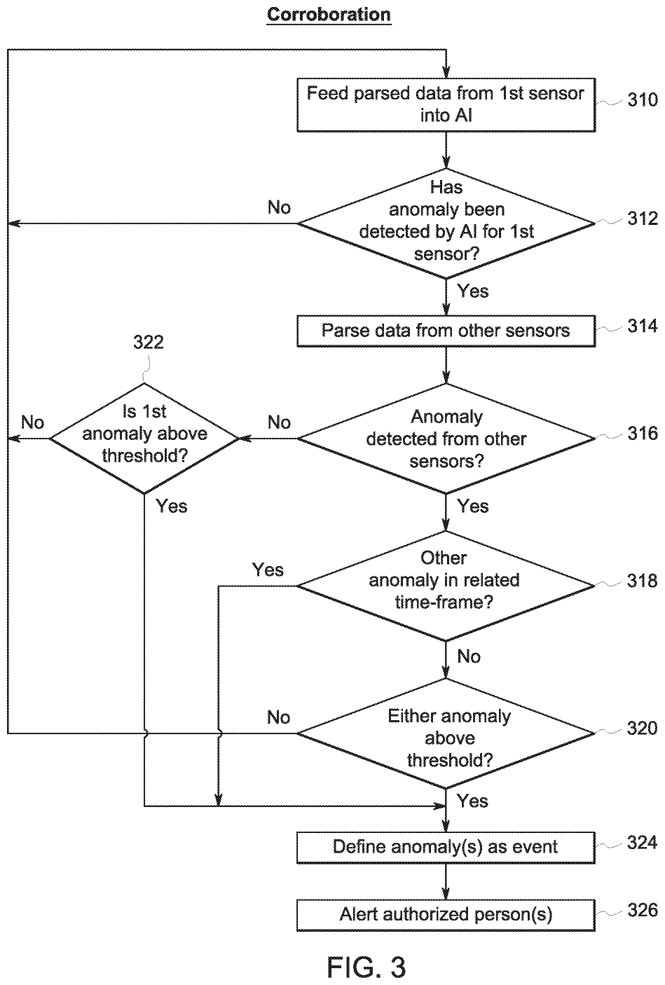

Another embodiment of the logic in making a determination, in this case, based on grading of an anomaly and/or corroboration between sensors is shown in FIG. 3.

Parsed data from a first sensor is fed into an AI system (step 310). Insofar as an anomaly is detected in the data (step 312), this is corroborated against data from at least one other sensor by parsing data from the other sensors that are involved in the particular implementation (step 314). In step 316 a decision is made whether any of the other sensor data shows up an anomaly, in which case it is compared on a time scale whether the second anomaly is in a related time frame (which could be the same time as the first sensor anomaly or be causally linked to activities flowing from the first sensor anomaly) (step 318). If the second sensor anomaly is above a first threshold deviation (step 320) and thus corroborates the first sensor, or similarly, even if there is no other corroborating sensor data, if the anomaly from the first or any other sensor data exceeds a second threshold deviation (step 322), the anomaly captured from either of such devices triggers an emergency event (step 324), which alerts one or more authorized persons (step 326).

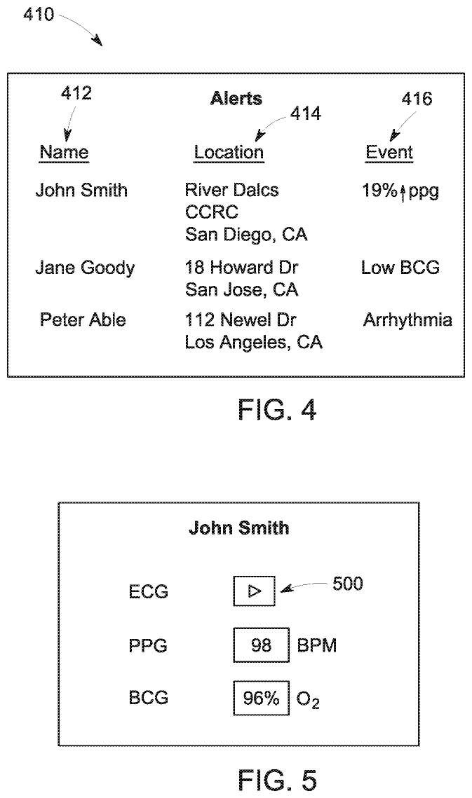

One embodiment of a dashboard for support personnel is shown in FIG. 4, and includes a list of flaggable events 410 from subjects supported by systems of the present implementation of the invention. Each flaggable event includes the name 412, location 414, and a short description 416 of the anomaly detected. By selecting any of the flaggable events, a detailed record of all sensor data for that subject, and time of the anomaly is presented as shown in the embodiment of FIG. 5 (ECG data 500 in this embodiment comprises a video clip of the ECG data for a 30 second time period starting from the time the anomaly was detected).

As mentioned above, corroboration of toilet seat sensor data can also be corroborated by data from non-toilet seat sensors.

In this embodiment, as shown in FIG. 1, the non-toilet seat sensors include a radar image capture device 160 mounted on a wall of the bathroom to detect any trigger events such as a fall by the subject. The non-toilet seat sensors also include an infra-red camera mounted outside the bathroom in the living room (not shown) of the subject for detecting the movement and gate of the subject as well as the subject's temperature in order to detect elevated temperatures and to validate the identity of the subject based on body configuration and gate of the person entering the bathroom. In this embodiment, the sensors also include a microphone 162 for detecting verbal and non-verbal sounds, e.g., calls for help or sounds of someone falling.

Studies have shown that the identity of a person can also be determined from anal images, much like a fingerprint. The present invention therefore also includes a secondary subject identity verification in the form of a camera 164 mounted inside the toilet bowl. In order to protect the privacy of the subject, the camera 164 includes a local processor and memory. The memory is configured with a control algorithm and defines a data store that stores previously recorded images of the subject's anal region to allow the processor to compare images captured by the camera 164 with images in the data store. The data transferred by the camera to the server 140 is thus limited to simply a confirmation of identity without any accompanying image data. This camera may also be used for observation of symptoms and/or diagnosis of conditions or progress thereof.

The present embodiment also includes a genetic sensor 166 for capturing genetic information of the subject, and a drug compliance sensor 168, for verifying subject compliance with the taking of medication, both of which are mounted in the toilet bowl for capturing information from stool and/or urine samples.

The sample may be analyzed immediately locally by on-board stool and/or urine analyzers, and the information about the sample and/or the analysis may be relayed to a remote party.

It will be appreciated that in order to identify an anomaly based on the data from as least some of these sensors, the database 142 will include base data captured from the particular subject or, where the parameters are generic, such as falling sounds, the base data may comprise generic third-party data.

In one embodiment, the algorithms for comparing data are implemented as an artificial intelligence (AI) system, e.g., artificial neural network, where base data is used as learning data for the AI system.

The present embodiment also includes micro-needles 170 that are recessed in the toilet seat and are only exposed once the subject is seated on the seat 110, in order to administer medication to the subject according to a user-specific dosage regimen and subject to confirmation of the identity of the subject.

To prevent scraping or scratching injury, the microneedle array may be recessed below the surface of the seat and momentarily deployed when the subject is immobile, and then immediately retracted once again.

In another embodiment, the seat 110 administers drugs via a dermal absorption jelly or ointment released from a reservoir in the seat 110 via a micro-pore array 180.

A similarly recessed microneedle or microneedle array may also be employed for the acquisition of blood samples and analyzed locally and information about the sample or the analysis relayed to a remote party, or the data or sample may be stored and preserved for collection by a courier for subsequent analysis.

While the present invention has been described with respect to specific embodiments, it will be appreciated that the invention could be implemented in different manners, with additional or different sensors and communication devices, and with differently configured processing of the data captured by the sensors, without departing from the scope of the invention.

The system may also include a voice-activated communication system for summoning help even when no sensor anomalies are detected. The communication system may also be used by response personnel to communicate with a subject when a flaggable event is received, in order to verify whether they are alright and also to allay their concerns if something is wrong, to let them know help is on the way.

What is claimed is:

1. A system for remote medicine, comprising
a smart toilet seat that includes at least one toilet seat sensor,
at least one non-toilet seat sensor,
a processor connected to the sensors,
memory enabled with machine readable code defining an algorithm for identifying a trigger event based on anomalies in the data from at least one toilet seat sensor, and defining an algorithm for corroborating the trigger event from the at least one toilet seat sensor with data from at least one other toilet seat sensor or at least one non-toilet seat sensor, to define a flagging event, and
notifying one or more people about the flagging event and details about said event.

2. The system of claim 1, wherein an anomaly includes one or more of a physiological characteristic or condition of interest, and a change in a physiological parameter.

3. The system of claim 1, wherein the at least one toilet seat sensor includes one or more of, an electrocardiogram (ECG) for measuring the electrical activity of the heart, a pulse oximeter or photoplethysmogram (PPG) for measuring blood oxygenation and localized pulse timing, a ballistocardiogram (BCG) for measuring the mechanical forces associated with the cardiac cycle, and a body weight sensor.

4. The system of claim 1, wherein the at least one non-toilet seat sensor includes one or more of an image capture device, a microphone, a genetic sensor, and a drug compliance sensor.

5. The system of claim 4, wherein the image capture device includes one or more cameras mounted at different locations and operating at different ranges of the frequency spectrum.

6. The system of claim 5, wherein the image capture device includes a camera mounted in the toilet for subject authentication.

7. The system of claim 1, wherein the algorithm for corroborating a trigger event includes logic for time stamping data from the sensors, wherein the corroboration of data includes an AI system configured to identify trigger events in the data from one or more of the sensors and comparing said data to data from one or more of the other sensors for a corresponding time frame.

8. The system of claim 1, wherein the smart toilet seat further comprises at least one of, means for administering medication or drugs to the subject, and means for capturing at least one of genetic data and drug compliance data from the subject.

9. The system of claim 8, wherein the means for administering of medication or drugs includes retractable micro-needles in the toilet seat, or a jelly or cream housed in a housing in the toilet seat with an active ingredient that is ejected to the surface of the seat through a micro-pore array, and is dermally absorbable.

10. A method of maintaining or improving the health of a subject, comprising
providing a subject's toilet with a smart toilet seat that includes one or more sensors for monitoring the physiological or psychological condition of the subject;
providing the subject or their living quarters with one or more sensors for monitoring the physiological or psychological condition of the subject;
detecting a trigger event in the data from at least one of the sensors based on anomalies compared to historical data from said subject or from other subjects, and comparing the trigger events to data from one or more of the other sensors to provide corroboration of a trigger event from at least one other sensor, and notifying one or more persons of a corroborated trigger event (also referred to herein as a flagging event).

11. The method of claim 10, wherein comparing the trigger event to data from at least one of the other sensors includes identifying the time frame of the trigger event and analyzing the data from at least one of the other sensors over a corresponding time frame or relevant adjacent timeframe.

12. The method of claim 10, further comprising capturing genetic data from the subject while the subject is sitting on the toilet seat.

13. The method of claim 12, further comprising analyzing the genetic data, including one or more of genomic sequencing, identifying specific genetic markers or sets of markers indicative of genetic predispositions, and identifying reactions or epigenetic changes in response to treatments.

14. The method of claim 10, further comprising administering medication to the subject while the subject is sitting on the toilet seat.

15. A method for providing telehealth to a subject, comprising
detecting physiological parameters in a subject using at least two sensors operating according to different modalities;
comparing the parameters of each sensor to previously captured data obtained from the subject to identify changes in the data to define a trigger event;
determining whether the changes exceed a pre-defined threshold to define a flagging event, and
comparing the trigger event identified from data from one sensor, which does not rise to the level of a flagging event to data from at least one other sensor for a corresponding time frame;
defining a flagging event based on corroboration of a trigger event, and
notifying one or more of: the subject, a designated contact person, a supervisory personnel, and a physician in the case of a flaggable event.

16. The method of claim 15, wherein the physiological parameters include at least one of patient weight, heart-related data, blood oxygenation data, and genetic data.

17. The method of claim 16, wherein the heart-related data includes one or more of blood pressure, heart rate, and Electrocardiogram (ECG) data.

18. The method of claim 16, wherein the heart-related data and weight are detected while the subject is sitting on a toilet, using sensors provided on the toilet seat.

19. The method of claim 15, wherein the identifying of changes and comparing of sensor data includes the use of an artificial intelligence (AI) system.

20. The method of claim 15, further comprising, verifying the identity of the subject.

21. The method of claim 15, further comprising administering medication to the subject while the subject is sitting on the toilet.
